(19) United States
(12) Patent Application Publication
(10) Pub. No.: US 2003/0035802 A1
(43) Pub. Date: Feb. 20, 2003

(54) Localization of A2B AdoR on human normal and diseased tissue: the use of anti-A2B antibody to diagnose and treat human tumors

(76) Inventors: Dewan Zeng, San Mateo, CA (US); Luiz Belardinelli, Menlo Park, CA (US); Maria B. Grant, Fairfield, FL (US)

Correspondence Address: Lisa M.W. Hillman, Ph.D, McDonnell Boehnen Hulbert & Berghoff, 32nd Floor, 300 S. Wacker Drive, Chicago, IL, 60606, US

(21) Appl. No.: 10/215,809

(22) Filed: Aug. 9, 2002

Related U.S. Application Data
(60) Provisional application No. 60/312,296, filed on Aug. 14, 2001.

(51) Int. Cl. A61K 51/00; A61K 39/395; G01N 33/574

(52) U.S. Cl. 424/146.1; 435/7.23; 424/1.49

(57) ABSTRACT

The invention provides methods of detection, prevention, amelioration, and treatment for angiogenesis, including angiogenesis associated with colon cancer and glioma. The methods comprise the use of an anti-A_{2B} antibody.

### TECHNICAL AREA OF THE INVENTION

The invention provides methods of detection, prevention, amelioration, and treatment for angiogenesis, including angiogenesis associated with colon cancer and glioma.

### BACKGROUND OF THE INVENTION

Angiogenesis is the generation of new blood vessels in a tissue or organ. Normally angiogenesis occurs only in very specific restricted situations, including, for example, wound healing, fetal and embryonal development, and formation of the corpus luteum, endometrium, and placenta.

Angiogenesis is controlled through a highly regulated system of angiogenic stimulators and inhibitors. The control of angiogenesis is altered in certain disease states and, in many cases, pathological damage associated with the disease is related to uncontrolled angiogenesis. Both controlled and uncontrolled angiogenesis likely proceed by the same mechanisms. Persistent, unregulated angiogenesis occurs in a variety of disease states, tumor metastases, and abnormal growth by endothelial cells.

Adenosine is released by hypoxic tissues and is believed to be an angiogenic factor that links altered cellular metabolism caused by O₂ deprivation to compensatory angiogenesis. Adenosine binds to four subtypes of G protein-coupled receptors termed A₁, A_{2A}, A_{2B} and A₃. It has been demonstrated that adenosine activation of the A_{2B} adenosine receptor (AdoR) increased cAMP accumulation, cell proliferation and VEGF expression in human retinal endothelial cells. It has been previously reported that the activation of A_{2B} AdoR increased vascular endothelial cell growth factor (VEGF) mRNA and protein expression in human retinal endothelial cells.

### DESCRIPTION OF THE DRAWINGS

FIGS. 1A-D shows glioma tissue sections from a human patient labeled with rabbit anti-A_{2B} antisera (FIG. 1A), rabbit pre-immune sera (FIG. 1C), and rabbit anti-vWF antibody (FIG. 1B). FIG. 1D shows a hematoxylin and eosin (H&E) stain of the glioma tissue section. No specific staining with rabbit pre-immune sera was observed. The anti-A_{2B} serum and anti-vWF labeled the capillaries of the glioma specimen.

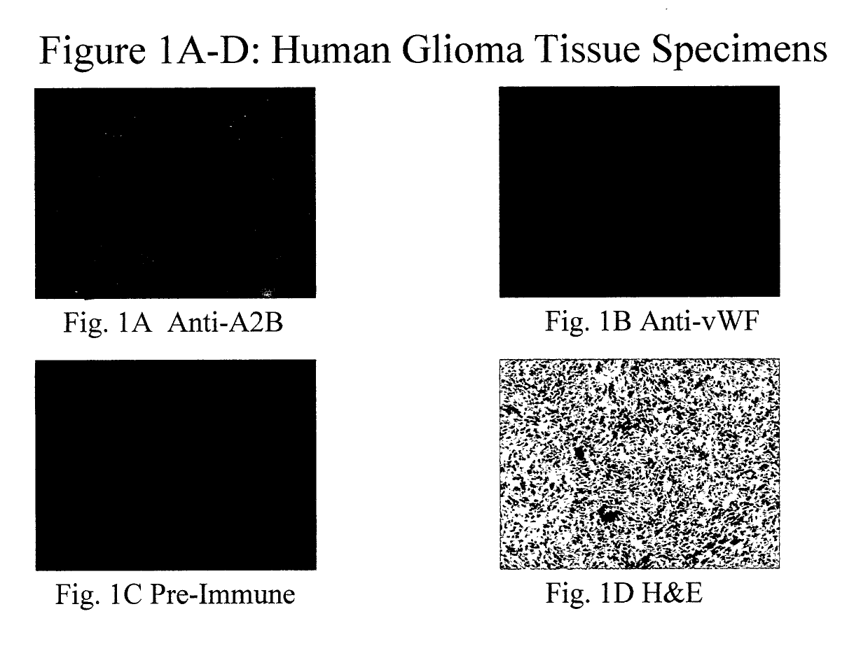

FIGS. 2A-C shows that anti-A_{2B} antiserum labels endothelial cells of a human glioma patient. A human glioma tissue section was labeled with rabbit anti-A_{2B} antiserum (secondary antibody: Cy 3-Goat anti-Rabbit IgG, red color) (FIG. 2A), mouse anti-vWF antibody (secondary antibody Alexa 488 Goat Anti-Mouse IgG, green color) (FIG. 2B), or double-labeled with both rabbit anti-A_{2B} antiserum and mouse anti-vWF antibody (FIG. 2C).

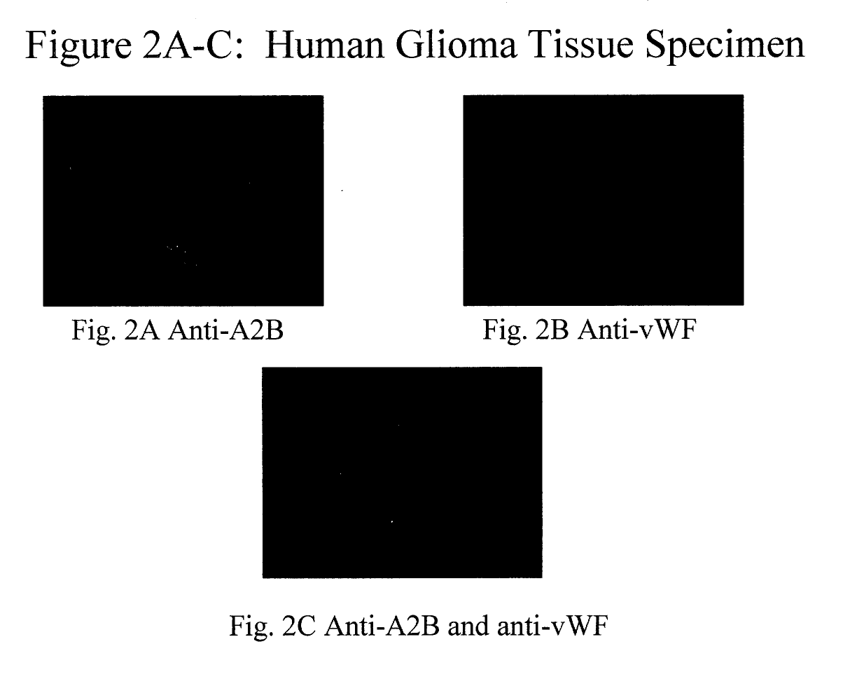

FIGS. 3A-C shows that anti-A_{2B} antiserum labels endothelial cells of a human glioma patient and this labeling can be completely blocked by the presence of the polypeptide that was used to raise the antibody. A human glioma patient section was labeled with rabbit anti-A_{2B} antiserum in the absence (FIG. 3A) or presence of the polypeptide that was used to raise the antibody (FIG. 3B). No specific staining with rabbit pre-immune serum was observed (FIG. 3C). FIG. 3D shows a hemotoxylin and eosin (H&E) stain of the adjacent glioma tissue section.

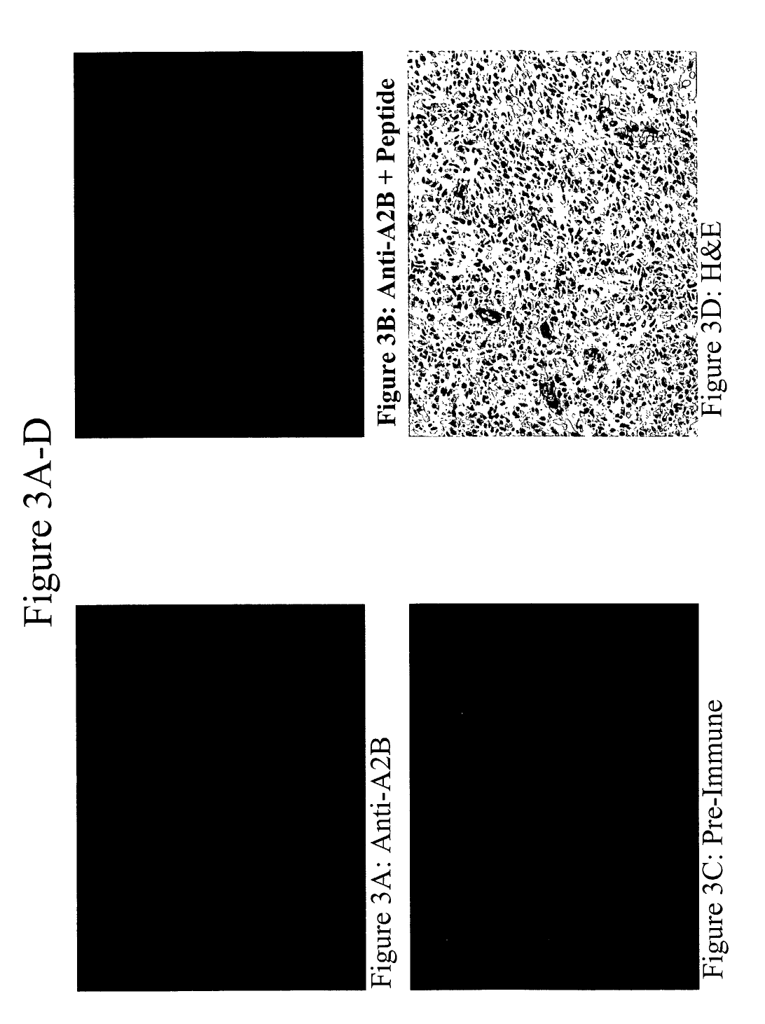

FIGS. 4A-D shows a human normal brain tissue section that was double-labeled with rabbit anti-A_{2B} antiserum (secondary antibody: Cy 3-Goat anti-Rabbit IgG, red color) (FIG. 4A) and mouse anti-vWF antibody (secondary antibody Alexa 488 Goat Anti-Mouse IgG, green color) (FIG. 4B), with rabbit pre-immune antiserum (secondary antibody: Cy3-goat anti rabbit IgG, red color) (FIG. 4C), or double-labeled with both rabbit anti-A_{2B} antiserum and mouse anti-vWF antibody (secondary antibody: Cy3-goat anti rabbit IgG, red color; secondary antibody: Alexa 488 Goat Anti-Mouse IgG, green color) (FIG. 4D). Compared to the pre-immune sera, there is no specific staining with anti-A2B anitsera in this brain section.

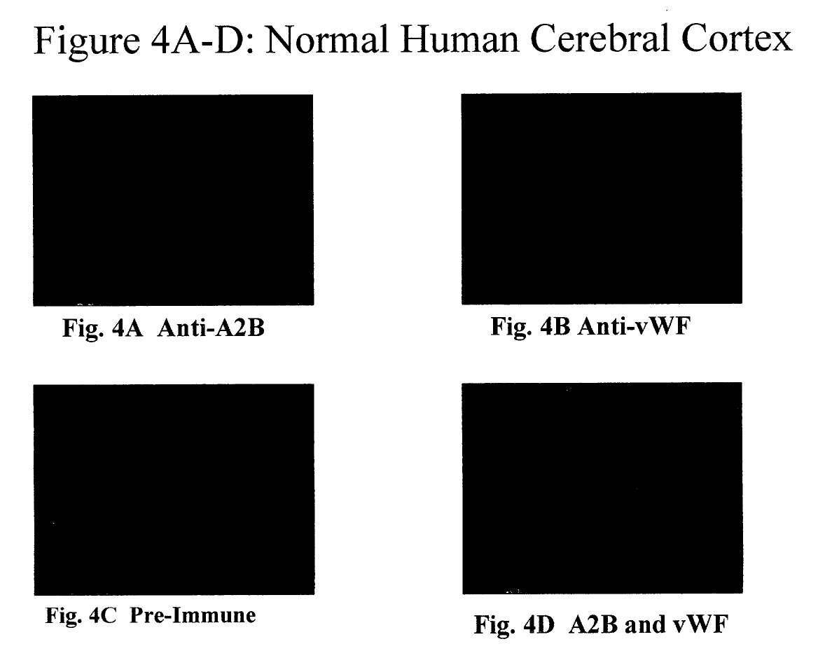

FIGS. 5A-D shows that anti-A_{2B} antiserum does not label endothelial cells of normal human kidney tissue. A normal human kidney tissue section was labeled with rabbit anti-A_{2B} antiserum (secondary antibody: Cy 3-Goat anti-Rabbit IgG, red color) (FIG. 5A), mouse anti-vWF antibody (secondary antibody Alexa 488 Goat Anti-Mouse IgG, green color) (FIG. 5B), or double-labeled with both rabbit anti-A_{2B} antiserum and mouse anti-vWF antibody (FIG. 5C). FIG. 5D show a H&E stain of the kidney tissue section.

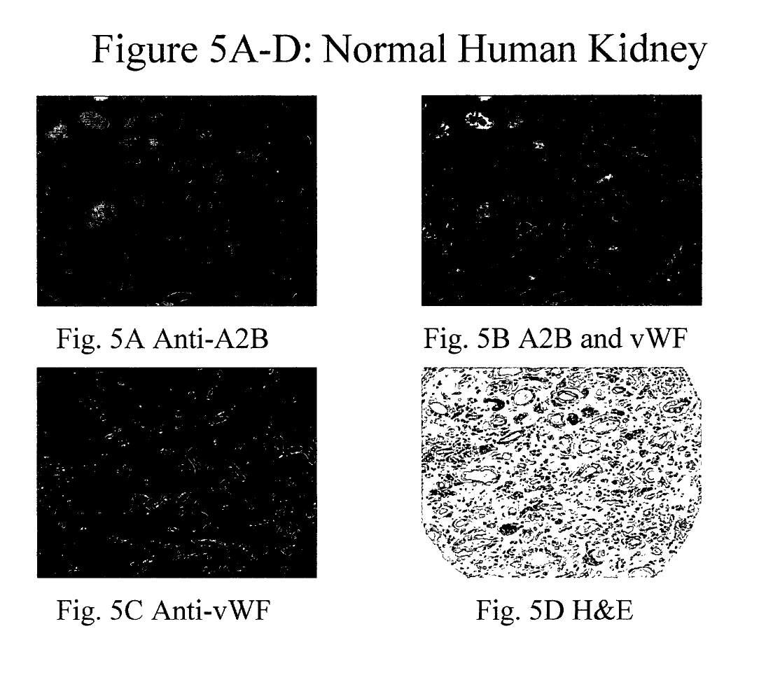

### SUMMARY OF THE INVENTION

It is an object of the invention to provide methods and compositions for the detection, treatment, amelioration, and prevention of angiogenesis, colon cancer, and glioma. This and other objects of the invention are provided by one Or more of the embodiments described below.

One embodiment of the invention provides a method of treating, preventing, or ameliorating angiogenesis in a mammal. The method comprises administering an anti-A_{2B} antibody to the mammal, whereby angiogenesis is treated, prevented, or ameliorated. The angiogenesis can be associated with a glioma or colon cancer. The mammal can be a human. The anti-A_{2B} antibody can be a monoclonal antibody or a polyclonal antibody. The antibody can be directed against an extracellular, an intracellular or a cytoplasmic tail region of an A_{2B} adenosine receptor. The antibody can be administered to the mammal parenterally. The antibody can also be conjugated to a cytotoxic agent or radioisotope.

Another embodiment of the invention provides a method of detecting the presence of angiogenesis in a mammal. The method comprises contacting a biological sample from the mammal with an anti-A_{2B} antibody that specifically binds an A_{2B} receptor under conditions that allow formation of an immunocomplex between the antibody and the A_{2B} receptor. A control sample is contacted with an anti-A_{2B} antibody that specifically binds an A_{2B} receptor under conditions that allow formation of an immunocomplex between the antibody and the A_{2B} receptor. Immunocomplexes are detected in both samples and detection of a greater amount of immunocomplexes in the sample from the mammal than in the control sample indicates the presence of angiogenesis in the mammal. The biological sample can be a tissue sample. The angiogenesis can be associated with a glioma or colon cancer.

Even another embodiment of the invention provides a method of detecting the presence of angiogenesis in a mammal, including, for example, a human. The method comprises contacting a tissue sample from the mammal with an anti-A_{2B} antibody that specifically binds an A_{2B} receptor under conditions that allow formation of an immunocomplex between the antibody and the A_{2B} receptor. Immunocomplexes are detected. Detection of immunocomplexes on capillaries of the tissue sample indicates the presence of angiogenesis. The angiogenesis can be associated with a glioma or colon cancer.

### DETAILED DESCRIPTION OF THE INVENTION

Antibodies

Antibodies of the invention are antibody molecules that specifically bind to an A_{2B} adenosine receptor or fragment thereof, but demonstrate little or no binding to non-A_{2B} adenosine receptor polypeptides. An A_{2B} adenosine receptor can be a mammalian receptor, for example, a human or other primate, mouse, rat, rabbit, guinea pig, goat, pig, cow, sheep, donkey, or a horse receptor.

In one embodiment of the invention an antibody specifically binds to an extracellular or intracellular region of A_{2B} adenosine receptor. For example, an antibody can bind to a cytoplasmic tail fragment of an A_{2B} adenosine receptor, including, for example, amino acids CQADVKSGNGQAGVQPALGVGL (SEQ ID NO:1) of a human A_{2B} adenosine receptor. An antibody directed against the polypeptide of SEQ ID NO:1 is specific for human A_{2B} adenosine receptor.

An antibody of the invention can be a polyclonal antibody, a monoclonal antibody, a single chain antibody (scFv), or a fragment of an antibody. Fragments of antibodies are a portion of an intact antibody comprising the antigen binding site or variable region of an intact antibody, wherein the portion is free of the constant heavy chain domains of the Fc region of the intact antibody. Examples of antibody fragments include Fab, Fab′, Fab′-SH, F(ab′)₂ and Fᵥ fragments. An antibody of the invention can be any antibody class, including for example, IgG, IgM, IgA, IgD and IgE.

An antibody or fragment thereof binds to an epitope of an A_{2B} adenosine receptor. An antibody can be made in vivo in suitable laboratory animals or in vitro using recombinant DNA techniques. Means for preparing and characterizing antibodies are well know in the art. See, e.g., Dean, Methods Mol Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhaln et al. Methods Cell. Biol. 37:7-56 (1993); Morrison, Ann. Rev. Immunol. 10:239-65 (1992); Wright et al. Crit. Rev. Immunol. 12:125-68 (1992). For example, polyclonal antibodies can be produced by administering an A_{2B} adenosine receptor polypeptide or fragment thereof to an animal, such as a human or other primate, mouse, rat, rabbit, goat, pig, cow, sheep, guinea pig, donkey, or horse. Serum from the immunized animal is collected and the antibodies are purified from the plasma by, for example, precipitation with ammonium sulfate, followed by chromatography, preferably affinity chromatography. Techniques for producing and processing polyclonal antibodies are known in the art.

Additionally, monoclonal antibodies directed against epitopes present on an A_{2B} adenosine receptor polypeptide or fragment thereof can be readily produced. Techniques for producing and processing monoclonal antibodies are known in the art. See e.g., Kohler & Milstein, Nature, 256:495 (1975). For example, normal B cells from a mammal, such as a mouse, which was immunized with an A_{2B} adenosine receptor polypeptide can be fused with, for example, HAT-sensitive mouse myeloma cells to produce hybridomas. Hybridomas producing A_{2B} adenosine receptor-specific antibodies can be identified using radioimmunoassay (RIA) or enzyme-linked immunosorbant (ELISA) and isolated by cloning in semi-solid agar or by limiting dilution. Clones producing A_{2B} adenosine receptor-specific antibodies are isolated by another round of screening. Therefore, monoclonal antibodies can be produced using a conventional hybridoma cell line, or by clones or subclones thereof or by cells carrying genetic information from the hybridoma cell line.

Particular isotypes of a monoclonal antibody can be prepared directly, by selecting from the initial fusion, or prepared secondarily, from a parental hybridoma secreting a monoclonal antibody of a different isotype by using a sib selection technique to isolate class-switch variants. See Steplewski et al., P.N.A.S. U.S.A. 82:8653 1985; Spria et al., J. Immunolog. Meth. 74:307, 1984. Monoclonal antibodies of the invention can also be recombinant monoclonal antibodies. See, e.g., U.S. Pat. No. 4,474,893; U.S. Pat. No. 4,816,567. Antibodies of the invention can also be chemically constructed. See, e.g., U.S. Pat. No. 4,676,980. Monoclonal antibodies can be screened for specificity using standard techniques, for example, by binding a polypeptide of the invention to a microtiter plate and measuring binding of the monoclonal antibody by an ELISA assay.

Antibodies of the invention can be chimeric (See e.g., U.S. Pat. No. 5,482,856), humanized (Jones et al., Nature 321:522 (1986); Reichmann et al., Nature 332:323 (1988); Presta, Curr. Op. Struct. Biol. 2:593 (1992); U.S. Pat. No. 5,530,101; U.S. Pat. No. 5,585,089), or human antibodies. Human antibodies can be made by, for example, direct immortilization, phage display, transgenic mice, or a Trimera methodology, see e.g., Reisener et al., Trends in Biotechnol. 16:242-246 (1998).

Antibodies, either monoclonal and polyclonal, which are directed against an A_{2B} adenosine receptor, are particularly useful for detecting the presence of A_{2B} adenosine receptor antigens in a sample, such as a tissue sample from a human. An immunoassay for an A_{2B} adenosine receptor antigen can utilize one antibody or several antibodies. An immunoassay for an A_{2B} adenosine receptor antigen can use, for example, a monoclonal antibody directed towards an A_{2B} adenosine receptor epitope, a combination of monoclonal antibodies directed towards epitopes of an A_{2B} adenosine receptor, polyclonal antibodies directed towards the same A_{2B} adenosine receptor antigen, or a combination of monoclonal and polyclonal antibodies. Immunoassay protocols can be based upon, for example, competition, direct reaction, or sandwich type assays using, for example, labeled antibody. Antibodies can be detected and/or quantified using for example, direct binding assays such as RIA, ELISA assays or western blot assays. Antibodies of the invention can be labeled with any type of label known in the art, including, for example, fluorescent, chemiluminescent, bioluminescent, enzyme, colloidal metal, and radioisotope labels.

Antibodies or the invention or fragments thereof can be bound to a support and used to detect the presence of an A_{2B} adenosine receptor antigen. Supports include, for example, glass, polystyrene, polypropylene, polyethylene, dextran, nylon, amylases, natural and modified celluloses, polyacrylamides, agaroses and magletite.

Another technique that can provide increased sensitivity comprises coupling an antibody to a low molecular weight hapten. The haptens can then be specifically detected by means of a second reaction. For example, it is common to use haptens such as biotin, which reacts with avidin, or dinitrophenyl, puridoxal, and fluorescein, which react with specific antihapten antibodies.

Polyclonal or monoclonal antibodies of the invention can further be used to isolate A_{2B} adenosine receptor antigens by immunoaffinity columns. The antibodies can be affixed to a solid support by, for example, adsorption or by covalent linkage so that the antibodies retain their immunoselective activity. Optionally, spacer groups can be included so that the antigen binding site of the antibody remains accessible. The immobilized antibodies can then be used to bind A_{2B} adenosine receptor antigens from a sample, such as a biological sample including, for example, tissue, serum, plasma, or blood. The bound A_{2B} adenosine receptor antigens are recovered from the column matrix by, for example, a change in pH.

Antibodies of the invention can also be used in immunolocalization studies to analyze the presence and distribution of a polypeptide of the invention during various cellular events or physiological conditions. Antibodies can also be used to identify molecules involved in passive immunization and to identify molecules involved in the biosynthesis of non-protein antigens. Identification of such molecules can be useful in vaccine development. Antibodies of the invention, including, for example, monoclonal antibodies and single chain antibodies, can be used to monitor the course of amelioration of a disease.

Methods of Detecting Angiogenesis

Diseases and conditions associated with increased release of adenosine, including for example, angiogenesis, glioma, and colon cancer can be detected using anti-A2B adenosine receptor antibodies. A glioma is a neuroectodermal tumor of neuroglial origin. Gliomas include, for example, astrocytoma, oligodendroglioma and ependymoma derived from astrocytes, oligodendrocytes and ependymal cells respectively. Gliomas infiltrate adjacent brain tissue, but in general they do not metastasize. Other examples of diseases associated with angiogenesis that can be detected, treated, ameliorated, or prevented according to the methods of the invention include, for example, Kaposi's sarcoma, hemangiomas, solid tumors, blood-borne tumors, breast cancer, lung cancer, ovarian cancer, testicular cancer, prostate cancer, rhabdomyosarcoma, retinoblastoma, Ewing's sarcoma, neuroblastoma, and osteosarcoma, diabetic retinopathy, macular degeneration, chronic uveitis/vitritis, retinopathy of prematurity, scleritis, pemphigoid, corneal graft rejection, neovascular glaucoma, retrolental fibroplasia, epidemic keratoconjunctivitis, infections causing retinitis or choroiditis, presumed ocular histoplasmosis, contact lens overwear, atopic keratitis, Terrien's marginal degeneration, marginal keratolysis, superior limbic keratitis, pterygium keratitis sicca, myopia, radial keratotomy, optic pits, chronic retinal detachment, hyperviscosity syndromes, trauma and post-laser complications associated with angiogenosis, rubeosis, and diseases caused by the abnormal proliferation of fibrovascular or fibrous tissue, toxoplasmosis, Stargardt's disease, pars planitis, Best's disease, Eales' disease, psoriasis, Lyme's disease, systemic lupus erythematosis, sickle cell anemia, syphilis, pseudoxanthoma elasticum, Paget's disease, vein occlusion, artery occlusion, carotid obstructive disease, Vitamin A deficiency, acquired immune deficiency syndrome, acne rosacca, phylectenulosis, Mycobacteria infections, lipid degeneration, chemical burns, Herpes simplex infections, Herpes zoster infections, protozoan infectionstrauma, rheumatoid arthritis, systemic lupus, rheumatoid arthritis, Osler-Weber-Rendu disease, polyarteritis, Wegener's disease, and Stevens-Johnson disease, ulcerative colitis, inflammatory bowel disease, Crohn's disease, Mooren's ulcer, Behcet's disease, Sjogrens disease, bacterial ulcers, fungal ulcers, and sarcoidosis.

Detection can be performed, by for example, contacting a biological sample from a mammal with an anti-A_{2B} antibody that specifically binds an A_{2B} receptor under conditions that allow formation of an immunocomplex between the antibody and the A_{2B} receptor. Detection can also be performed in vivo. Immunocomplexes can be detected using any method known in the art. In one embodiment of the invention a control sample can also be contacted with an anti-A_{2B} antibody that specifically binds an A_{2B} receptor under conditions that allow formation of an immunocomplex between the antibody and the A_{2B} receptor. Detection of a greater amount of immunocomplexes in the sample from the mammal than in the control sample indicates the presence of a disease or disease condition associated with angiogenesis.

In one embodiment of the invention, detection of the location of the immunocomplexes in a biological sample, such as a tissue sample, can be used to determine the presence of angiogenesis, including angiogenesis associated with a glioma, or colon cancer in a mammal. For example, a tissue sample from a mammal can be contacted with an anti-A_{2B} antibody that specifically binds an A_{2B} receptor under conditions that allow formation of an immunocomplex between the antibody and the A_{2B} receptor. Detection of immunocomplexes on capillaries of the sample indicates that the mammal has angiogenesis.

Preferably, the tissue to be assayed will be obtained by surgical procedures, e.g., biopsy. The excised tissue will be assayed for the presence of an antigen that recognizes an anti-A_{2B} adenosine receptor antibody as described above, by methods generally known in the art, including, for example, imunohistochemistry, RIA, ELISA, and immobilized immunoassays. Tissue can be fixed or frozen to permit histological sectioning, and can be stained prior to incubation with the antibody. An antibody can be labeled, for example with a dye or fluorescent label, chemical label, heavy metal label, chemiluminescent label, bioluminescent label, enzyme label, or radioactive label to permit the detection and localization of the antibody in the assayed tissue. A radioactive label can be for example, radioiodine, indium-111, gallium-67, technetium-99m, or a positron emitting radioisotope.

Alternatively, anti-A_{2B} antibodies can be used for in vivo detection of A_{2B} receptors. A labeled antibody is given to a mammal in a dose that is diagnostically effective. The term “diagnostically effective” means that the amount of labeled antibody is administered in sufficient quantity to enable detection of A_{2B} receptors in vivo.

The concentration of labeled antibody that is adminstered should be sufficient such that the binding to cells having A_{2B} receptors is detectable compared to background signals. The labeled antibody should be rapidly cleared from the circulatory system in order to give the best target-to-background signal ratio. The dosage of labeled antibody for in vivo diagnosis will vary depending on such factors as age, sex, and extent of disease of the individual. Dosages can vary, for example, depending on whether multiple injections are given and antigenic burden.

An antibody for in vivo detection of A_{2B} receptors is preferably labeled with a radioisotope. The type of detection instrument used can influence the selection of a radioisotope. A radioisotope should have a type of decay which is detectable for a given type of instrument. A radioisotope should be selected so that deleterious radiation to the mammal is minimized. Preferably, a radioisotope used for in vivo imaging will lack a partical emission, but produce a large number of photons in the 140-250 keV range, which may readily be detected by conventional gamma cameras.

For in vivo detection, radioisotopes can be bound to an antibody either directly or indirectly by using an intermediate functional group. Intermediate functional groups include, for example, bifunctional chelating agents such as diethylenetriaminepentacetic acid (DTPA), ethylenediaminetetraacetic acid (EDTA) and similar molecules. Typical examples of metallic ions which can be bound antibodies of the invention include ¹¹¹In, ⁹⁷Ru, ⁶⁷Ga, ⁶⁸Ga, ⁷²As, ⁸⁹Zr, and ²⁰¹Tl.

Anti-A_{2B} antibodies can also be labeled with a paramagnetic isotope for in vivo detection, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include, for example, ¹⁵⁷Gd, ⁵⁵M, ¹⁶²Dy, ⁵²Cr, and ⁵⁶Fe.

Anti-A_{2B} antibodies can also be used in vitro and in vivo to monitor the course of amelioration of a disease in a patient. For example, by measuring changes in the amount of immunocomplexes comprising an anti-A_{2B} antibody and an A_{2B} receptor present in a tissue or patient it would be possible to determine whether a particular therapeutic regimen aimed at ameliorating a disease associated with angiogenesis, colon cancer, or glioma is effective.

Methods of Treatment, Amelioration, or Prevention

Anti-A_{2B} adenosine receptor antibodies can be used to treat, ameliorate, or prevent diseases or conditions, for example, angiogenesis and cancer including glioma and colon cancer. Treatment, amelioration, or prevention can be effected by an antibody, such as an anti-A_{2B} adenosine receptor monoclonal antibody or fragments thereof, which is administered to an animal, such as a human. In one embodiment of the invention an antibody or fragment thereof is administered to an animal in a pharmaceutical composition comprising a pharmaceutically acceptable carrier. A pharmaceutical composition comprises a therapeutically effective amount of an antibody or fragments thereof. A therapeutically effective amount of an antibody or fragments thereof is an amount effective in treating, ameliorating, or preventing angiogenesis and cancer such as glioma or colon cancer.

In another aspect of the invention, therapeutic agents, for example radioisotope or cytotoxic agents can be conjugated to an anti-A_{2B} antibody of the invention. See e.g., Reisfeld et al. Antibodies as Therapeutic Agents and Carriers for Drugs, Permagon Press, 1990. A radioisotope can be, for example, a beta-emitting radioisotope such as iodine-131 or yttrium-90, or an alpha or auger emitting radioisotope such as bismuth-212 or astatine. See e.g., Therapeutic Monoclonal Antibodies, C. Borrebaeck et al. eds., 1990, M. Stockton Press. An antibody of the invention can also be conjugated to a cytotoxic agent such as a chemotherapeutic drug, a biologic toxin, or an enzyme. A biologic toxin can be, for example, bryodin, ricin, idarubicin, abrin, amantin, saporin, gelonin, diphtheria toxin, pseudomonas exotoxin A, trichosanthin, restrictocin, or mycotoxin. An enzyme can be, for example, carboxypeptidase, alkaline phosphatase, or thymidine kinase. See e.g., Monoclonal Antibodies: Production, Engineering, and Clinical Applications, M. A. Ritter et al., eds. Cambridge University Press, 1995.

An anti-A_{2B} adenosine receptor antibody of the invention can be administered to a mammal, such as a mouse, rabbit, guinea pig, macaque, baboon, chimpanzee, human, cow, sheep, pig, horse, dog or cat.

Administration of an antibody can be by any means known in the art, including intramuscular, intravenous, intrapulmonary, intramuscular, intradermal, intraperitoneal, or subcutaneous injection, intranasal, aerosol, infusion pump, suppository, mucosal, topical, and oral. Preferably, an antibody is accompanied by a protein carrier for oral administration. A combination of administration methods can also be used.

Pharmaceutically acceptable carriers and diluents for therapeutic use are well known in the art and are described in, for example, Remington's Pharmaceutical Sciences, Mack Publishing Co. (A. R. Gennaro ed. (1985)). The carrier should not itself induce the production of antibodies harmful to the host. Such carriers include, but are not limited to, large, slowly metabolized, macromolecules, such as proteins, polysaccharides such as latex functionalized sepharose, agarose, cellulose, cellulose beads and the like, polylactic acids, polyglycolic acids, polymeric amino acids such as polyglutamic acid, polylysine, and the like, amino acid copolymers, peptoids, lipitoids, and inactive, avirulent virus particles or bacterial cells. Liposomes, hydrogels, cyclodextrins, biodegradable nanocapsules, and bioadhesives can also be used as a carrier for a composition of the invention.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, phosphate buffered saline, glycerol, glucose, dextrose, malodextrin, ethanol, Ringer's solution, Hank's solution, or the like, singly or in combination, as well as substances such as wetting agents, tonicity adjusting agents, detergent, emulsifying agents, or pH buffering agents. Additional active agents such as bactericidal agents can also be added.

The compositions of the invention can be formulated into ingestible tablets, buccal tablets, troches, capsules, elixirs, suspensions, syrups, wafers, injectable formulations, and the like. The percentage of antibodies in such compositions and preparations can vary from 0.1% to 60% of the weight of the unit.

Antibodies can be administered either to a mammal that does not have symptoms of angiogenesis, including angiogenesis associated glioma or colon cancer or can be administered to a mammal having angiogenesis, glioma, or colon cancer. The particular dosages of antibodies in a composition will depend on many factors including, but not limited to the species, age, gender, concurrent medication, general condition of the mammal to which the composition is administered, and the mode of administration of the composition. An effective amount of the composition of the invention can be readily determined using only routine experimentation. In one embodiment of the invention an antibody can be administered to a mammal in a dose of about 1-100 mg/kg/day. In another embodiment of the invention an antibody can be administered to a mammal in a dose of about 1-10 mg/kg/day.

The materials for use in a method of the invention can be present in a kit. A kit can comprise one or more elements used in the method. For example, a kit can contain an antibody of the invention in a container and A_{2B} adenosine receptor polypeptides in another container. The kit and containers are labeled with their contents and the kit includes instructions for use of the elements in the containers. The constituents of the kit can be present in, for example, liquid or lypholized form.

The following are provided for exemplification purpose only and are not intended to limit the scope of the invention described in broad terms above. All references cited in this disclosure are incorporated herein by reference.

### EXAMPLES

### Example 1

The cellular localization of an A_{2B} adenosine receptor in human diseases characterized by abnormal angiogenesis was determined. A polyclonal antibody against the cytoplasmic tail of human A_{2B} adenosine receptor (CQADVKSGNGQAGVQPAL GVGL; SEQ ID NO:1) was raised in rabbit and characterized in vitro.

Human tissues from glioma and normal tissues were obtained and formalin-fixed, paraffin-embedded, cut into thin sections and mounted onto glass slides. These sections were treated with xylene (twice, 3 minutes each), ethanol (100% and 95%) and water to remove the wax. To retrieve antigen, the sections were boiled for 1 hour in 10 mM sodium citrate (pH to 6.0 with citric acid) and washed twice in PBS. After blocking with PBS (pH=7.4) with 0.25% Triton X-100 and 10% goat serum for 1 hour, the sections were incubated overnight in a humidified chamber with primary antibodies (rabbit anti-A_{2B} antiserum and mouse anti-van Willebrand Factor antibody (anti-vWF; a marker for endothelial cells). After washing five times with PBS, the sections were blocked with again in PBS (7.4) with 0.25% Triton X-100 and 10% goat serum for 30 minutes and then were incubated with fluorescence conjugated-secondary antibodies (Cy 3-Goat anti-Rabbit IgG and Alexa 488 Goat Anti-Mouse IgG) for 4 hours. The sections were washed trice in PBS and mounted with Vector aqueous mounting medium with anti-fading agents. Tissue sections were examined at 200× magnification using a Nikon ECLIPSE (TE300) microscope and digital images were obtained using SPOT software.

The sections were single-labeled with rabbit anti-A_{2B} antisera, rabbit pre-immune sera, or rabbit anti-vWF antibody. No specific staining with rabbit pre-immune sera was observed. The anti-A_{2B} serum labeled the capillaries of the glioma specimen, but did not label the capillaries of normal brain. In contrast, ant-vWF antibody labeled the capillaries of both normal and glioma tissues. Therefore, immunoreactivity with A_{2B} adenosine receptor was detected on the capillaries of human glioma, suggesting that A_{2B} adenosine receptor is up-regulated and mediates the effect of adenosine in stimulating angiogenesis in disease.

We claim:

1. A method of treating, preventing, or ameliorating angiogenesis in a mammal comprising administering a specific anti-A_{2B} antibody to the mammal, whereby angiogenesis is treated, prevented, or ameliorated.

2. The method of claim 1, wherein the angiogenesis is associated with glioma.

3. The method of claim 1, wherein the angiogenesis is associated with colon cancer.

4. The method of claim 1, wherein the mammal is a human.

5. The method of claim 1, wherein the anti-A_{2B} antibody is a monoclonal antibody.

6. The method of claim 1, wherein the anti-A_{2B} antibody is a polyclonal antibody.

7. The method of claim 1, wherein the antibody is directed against an extracellular or an intracellular region of an A_{2B} adenosine receptor.

8. The method of claim 1, wherein the antibody is directed against cytoplasmic tail of an A_{2B} adenosine receptor.

9. The method of claim 1, wherein the antibody is administered to the mammal parenterally.

10. The method of claim 1, wherein the antibody is conjugated to a cytotoxic agent or radioisotope.

11. A method of detecting the presence of angiogenesis in a mammal comprising:
contacting a biological sample from the mammal with an anti-A_{2B} antibody that specifically binds an A_{2B} receptor under conditions that allow formation of an immunocomplex between the antibody and the A_{2B} receptor;
contacting a control sample with an anti-A_{2B} antibody that specifically binds an A_{2B} receptor under conditions that allow formation of an immunocomplex between the antibody and the A_{2B} receptor; and
detecting immunocomplexes in both samples,
wherein detection of a greater amount of immunocomplexes in the sample from the mammal than in the control sample indicates the presence of angiogenesis in the mammal.

12. The method of claim 11, wherein the biological sample is a tissue sample.

13. The method of claim 11, wherein the antibody is polyclonal.

14. The method of claim 11, wherein the antibody is monoclonal.

15. The method of claim 11, wherein the angiogenesis is associated with a glioma.

16. The method of claim 11, wherein the angiogenesis is associated with colon cancer.

17. The method of claim 11, wherein the mammal is a human.

18. A method of detecting the presence of angiogenesis in a mammal comprising:
contacting a tissue sample from the mammal with an anti-A_{2B} antibody that specifically binds an A_{2B} receptor under conditions that allow formation of an immunocomplex between the antibody and the A_{2B} receptor; and
detecting immunocomplexes;
wherein, the detection of immunocomplexes on capillaries of the tissue sample indicates the presence of angiogenesis.

19. The method of claim 18, wherein the antibody is polyclonal.

20. The method of claim 18, wherein the antibody is monoclonal.

21. The method of claim 18, wherein the angiogenesis is associated with a glioma.

22. The method of claim 18, wherein the angiogenesis is associated with colon cancer.

23. The method of claim 18, wherein the mammal is a human.
